(19) United States
(12) Patent Application Publication
(10) Pub. No.: US 2019/0117840 A1
(43) Pub. Date: Apr. 25, 2019

(54) TISSUE SCAFFOLDS

(71) Applicant: NORTHWICK PARK INSTITUTE FOR MEDICAL RESEARCH LTD., Harrow, Greater London (GB)

(72) Inventors: Tahera Iqbal ANSARI, Harrow, Greater London (GB); Karin GRECO, Harrow, Greater London (GB)

(21) Appl. No.: 16/091,315

(22) PCT Filed: Mar. 30, 2017
(86) PCT No.: PCT/GB2017/050897

§ 371 Date: Oct. 4, 2018

(30) Foreign Application Priority Data
Apr. 5, 2016 (GB) 1605782.0

(51) Int. Cl. A61L 27/36; A61L 27/50

(52) U.S. Cl. CPC A61L 27/3687; A61L 27/50; A61L 27/362; A61L 27/3691

(57) ABSTRACT

This invention provides cross-linked dermal scaffolds, dermal replacements and methods for making the same in which particles or granules of biological tissue are cross-linked with quercetin and/or genipin. The cross-linked tissue is prepared by decellularising biological tissue and cross-linking the biological tissue with quercetin and/or genipin, with the tissue being grown into particles or granules either before or after cross-linking.

### TECHNICAL FIELD OF THE INVENTION

This invention relates to tissue scaffolds, bulking agents (e.g. sphincters), dermal replacements and methods of manufacturing the same. In particular, the invention relates to cross-linked dermal scaffolds, dermal replacements and methods for making the same.

### BACKGROUND TO THE INVENTION

Tissue engineering includes the construction of materials to repair and regenerate a variety of tissue defects and organs through a combination of cells, scaffolds and biomolecules in existence for the past twenty years. Designed as a skin substitute, both epidermal and dermal acellular scaffolds have been under investigation for over a decade and it is known to use a non-enzymatic decellularisation method to produce an acellular dermal scaffold in less than two days. The extracellular matrix (ECM) components of such scaffolds can be preserved, which help to support cell migration, proliferation and differentiation when the scaffold is implanted or adhered to biological tissue. ECM is highly conserved among species and consists of molecules such as collagen, fibronectin, laminin, vitronectin, glycosaminoglycans (GAG), and growth factors. Collagen is the primary mechanostructural element for dermis, conferring tensile strength and proteolytic resistance to the tissue. Although natural cross-linking of collagen occurs, the chemicals used for decellularisation are believed to loosen the collagen fibrils and disrupt the microstructure of the ECM, which makes it liable to degradation by the collagenase in the host organism. Hence, it is often necessary to confer structural stability and collagenase resistance to implanted materials by the introduction of exogenous cross-linking into the molecular structure, which can change in intensity and degree depending on the final biomedical application. Exogenous crosslinking agents stabilize the collagen molecule by forming covalent and hydrogen bonds between the fibres. Many different cross-linking agents are available, including well-known and well-used compounds such as glutaraldehyde, for example.

Genipin is an iridoid glycoside, one of the main ingredients extracted from, for example, the gardenia fruit (Gardenia jasminoides) and has previously been used to crosslink biological matrices in the production of porcine dermal sheets, as described in Greco et al. Journal of Biomaterials Applications, 2015, Vol, 30(2) 239-253. Genipin has certain advantages over many chemical cross-linking agents, for example, lower levels of cytotoxicity compared to glutaraldehyde, and it is cytocompatible as well as resistant to biodegradation. However, genipin is relatively expensive when compared to some crosslinking agents, and it is capable of forming strong crosslinking bonds with collagen which can result in it being retained in the fibre structure of collagen scaffolds for over three months, which may not be desirable for certain applications. For certain temporary biomedical applications, milder crosslinking might be required, such as for restoration of non-healing skin ulcers where it might be necessary to have a biodegradable, yet stable scaffold to support tissue reconstruction, including reepithelialisation.

It would therefore be advantageous to provide alternative cross-linking agents for dermal scaffolds, which mitigate or avoid the problems of the prior art cross-linking agents, including having low cytotoxicity compared to chemical agents such as glutaraldehyde and hexamethylene diisocyanate (HMDI). It would also be advantageous to provide alternative cross-linking agents to genipin which are more cost-effective and which can be used to create looser cross-linking bonds than those produced using genipin, in order to produce a scaffold having lower stiffness for use in restoring non-healing skin ulcers, for example.

In addition, it would be advantageous to produce scaffolds, such as dermal scaffolds, using genipin or other low cytotoxic cross-linking agents in which the scaffold is in a form which is flexible enough to mould about a damaged tissue without requiring significant manipulation, or which adheres to the shape and configuration of the site.

It is therefore an aim of embodiments of the invention to overcome one or more problems of the prior art.

### SUMMARY OF THE INVENTION

According to a first aspect of the invention there is provided a tissue scaffold comprising decellularised dermal tissue cross-linked with quercetin.

Quercetin is a flavonol found in many fruits, vegetables, leaves and grains, for example from the Quercus species of trees (oak) and capers. It may be dehydrated, in the monohydrate or dihydrate form.

The resultant scaffold provides an excellent material for the repair and regeneration of dermal tissue.

The dermal tissue may comprise epidermis, dermis, hypodermis, basement membrane or any combination thereof, for example. In preferred embodiments the dermal tissue comprises both epidermis and dermis, and is preferably intact skin.

In some embodiments the decellularised tissue comprises at least 50%, 55%, 60%, 65%, 70% or at least 75% of the extracellular matrix (ECM) proteins present in the tissue prior to decellularisation.

In some embodiments the decellularised tissue comprises at least 75%, 80%, 85% or at least 90% of the collagens present in the tissue prior to decellularisation.

The degree of cross-linking in the cross-linked tissue may be at least 5%, 7.5%, 10%, 12.5%, 15%, 17.5% or at least 20%. The degree of cross-linking in the cross-linked tissue may be no more than 65%, 60%, 55%, 50% or no more than 45%. In particularly preferred embodiments the degree of cross-linking may be no more than 42.5%, 40%, 37.5%, 35%, 32.5% or no more than 30% and may be between 10% and 50%, between 15% and 40% or between 20% and 30%, for example. The degree of cross-linking may be determined using a ninhydrin assay, for example.

By “decellularised” we mean that at least 95%, 96%, 97%, 98%, 99%, 99.5, 99.6%, 99.7%, 99.8%, 99.9% or substantially all cells have been removed from the tissue.

The quercetin may comprise unhydrated quercetin or a hydrate of quercetin, such as the monohydrate or dihydrate.

According to a second aspect of the invention there is provided a tissue implant comprising a tissue scaffold of the first aspect of the invention.

According to a third aspect of the invention there is provided a dermal tissue replacement comprising a tissue scaffold of the first aspect of the invention.

According to a fourth aspect of the invention there is provided a method of manufacturing a dermal tissue scaffold of the first aspect of the invention the method comprising the steps of:

a) decellularising dermal tissue, and

b) cross-linking the decellularised tissue with quercetin.

Each of the dermal tissue and quercetin may be as described hereinabove for the first to third aspects of the invention.

Step (a) may comprise subjecting the dermal tissue to a decellularisation process comprising subjecting the tissue to osmotic shock. The decellularisation may comprise contacting the tissue sequentially with hypotonic and hypertonic solutions (in any order) to promote cell lysis.

It has been found that decellularisation using osmotic shock treatment is particularly effective for subsequent cross-linking with quercetin, and for enabling grinding or milling of the decellularised tissue, if desired.

The dermal tissue may be immersed in the hypotonic and hypertonic solutions.

Contact, or immersion, of the dermal tissue with the hypotonic solutions may be repeated at least once, and preferably at least twice, three times or four times.

The hypertonic solution may comprise sodium chloride. The hypertonic solution may further comprise ethylenediaminetetracetate (EDTA) and/or Tris-HCl.

The hypertonic solution may comprise between 0.5M and 2M NaCl, such as around 1M NaCl. The EDTA may be present at a concentration of between 10 mM and 100 mM, such as between 20 mM and 50 mM or around 25 mM. The Tris-HCl may be present at a concentration of between 20 mM and 100 mM, such as between 25 mM and 75 mM or around 50 mM.

The hypertonic solution may comprise 1M sodium chloride, and optionally 25 mM EDTA and 50 mM Tris-HCl.

The hypotonic solution may comprise EDTA and/or Tris-HCl which may be present at concentrations as described for the hypertonic solution described above.

The decellularisation process may also comprise contacting the dermal tissue with one or more nuclease. The nuclease may be contacted with the dermal tissue after the dermal tissue is contacted with the hypotonic and hypertonic solutions. The nuclease may be a DNase or an RNase or a combination of a DNase and RNase. The decellularisation process may also comprise washing the tissue, preferably in a saline solution, such as phosphate buffered saline (PBS), after each step.

Step (b) may comprise contacting the decellularised tissue with quercetin at a temperature of at least 5° C., 10° C., 15° C. or 20° C., preferably ambient temperature, more preferably between 15° C. and 25° C.

Step (b) may comprise contacting the decellularised tissue with an aqueous solution of quercetin. The quercetin may be present in the aqueous solution at a concentration of at least 0.1 mg/ml, 0.2 mg/ml, 0.3 mg/ml or at least 0.5 mg/ml, such as at least 1 mg/ml or above.

Step (b) may comprise contacting the decellularised tissue with quercetin for a time period of at least 5 minutes, 10 minutes, 15 minutes, 30 minutes, 60 minutes, 120 minutes or 180 minutes.

The aqueous solution of quercetin may comprise further ingredients such as co-solvent, buffer components or the like, for example. The quercetin may comprise quercetin in an ethanolic solution. The ethanolic solution may comprise at least 20% v/v ethanol, or at least 30% v/v ethanol or at least 40% v/v ethanol. In alternative embodiments the quercetin may be present in a saline solution, such as phosphate buffered saline (PBS) for example.

The decellularised dermal tissue may be immersed in a solution of quercetin. The ratio of the volume of quercetin solution and to the surface area of the decellularised tissue may be at least 0.1 ml/cm², at least 0.2 ml/cm² or at least 0.5 ml/cm².

There may be a step (c), after step (b), of washing the cross-linked tissue. Step (c) may comprise immersing the cross-linked tissue in a washing medium for at least 5 minutes, at least 10 minutes or at least 15 minutes. The cross-linked tissue may be subject to more than one washing step and step (c) may be repeated at least twice or three times. The washing medium may comprise a saline solution, such as PBS. The washing medium may comprise a preservative such as sodium azide, for example.

The biological tissue may be in the form of a sheet or piece of tissue. The sheet or piece of tissue may comprise dimensions of at least 1 cm×1 cm, 1 cm×2 cm, 2 cm×2 cm, 3 cm×3 cm or at least 4 cm×4 cm, for example.

In preferred embodiments the biological tissue may be in the form of granules or particles. The granules or particles may have an average diameter or length of at least 1 μm, 2 μm, 3 μm, 4 μm, 5 μm, 10 μm, 20 μm, 50 μm, 75 μm or 100 μm. In some embodiments the granules or particles may have an average diameter or length of 10 μm to 500 μm, 25 μm to 500 μm, 50 μm to 500 μm, 100 μm to 500 μm or 100 μm to 250 μm. The average particle size (diameter or length) may be measured using histological sections stained with Picro-sirius Red (PSR) and a graticule, or any other suitable method known to persons skilled in the art. The granules or particles may be in the form of a paste. The particles or granules may be formed by grinding (including freezing followed by grinding) or cryo-milling the cross-linked biological tissue into particles or granules.

The paste may have a viscosity of between 100 cP and 30×10⁶ cP, such as between 2500 cP and 30×10⁶ cP, 10,000 cP and 30×10⁶ cP, 100,000 cP and 30×10⁶ cP or between 1×10⁶ cP and 30×10⁶ cP.

The decellularised biological tissue may be converted from one physical form to another after step (a), such as converting it from a sheet or piece to granules or particles, for example.

Alternatively the decellularised tissue may be converted from one physical form to another after step (b), again such as conversion from a sheet or piece to granules or particles, for example.

There may be a step of forming a suspension of the cross-linked decellularised particles or granules in a carrier liquid.

The step of forming a suspension may comprise grinding (including freezing followed by grinding) or cryo-milling the cross-linked biological tissue into particles or granules, in embodiments where the dermal tissue is a sheet or pieces, followed by suspending the particles or granules in the carrier liquid. The carrier liquid may be water or an aqueous solution such as an ethanolic aqueous solution, or a buffer solution (such as PBS), for example.

The resultant suspension may comprise a paste. The paste may have a viscosity of between 100 cP and 30×10⁶ cP, such as between 2500 cP and 30×10⁶ cP, 10,000 cP and 30×10⁶ cP, 100,000 cP and 30×10⁶ cP or between 1×10⁶ cP and 30×10⁶ cP.

The suspension may comprise at least 25% wt biological tissue, at least 50% wt, at least 60%, at least 70% wt or at least 75% wt biological tissue. In some embodiments, when the suspension comprises a paste, for example, there may be at least 80% wt, 82% wt, 84% wt, 86% wt, 88% wt or at least 90% wt biological tissue.

According to a fifth aspect of the present invention there is provided a tissue scaffold comprising particles or granules of biological tissue, wherein the biological tissue is cross-linked with quercetin or genipin.

The quercetin may be as described hereinabove for the first to fourth aspects of the invention.

The biological tissue may be a dermal tissue as described hereinabove for the first to fourth aspects of the invention. Alternatively, the biological tissue may be non-dermal tissue, but is preferably dermal tissue. Suitable non-dermal tissues may be selected from interstitial, connective or supporting tissue, which may be cartilaginous, fibrocartilaginous or calcified cartilaginous tissue, for example bowel, trachea, oesophagus, blood vessel, stomach, urethra, bladder, lung, liver, spleen, kidney, larynx, synovial membrane, tendon, bone-tendon, bone-ligament or ligament, muscle for example.

The granules or particles may comprise a paste. The paste may be as described above for the first to fourth aspect of the invention, and may be formed by grinding or cryo-milling biological tissue.

The paste may have a viscosity of between 100 cP and 30×10⁶ cP, such as between 2500 cP and 3×10⁶ cP, such as between 2500 cP and 30×10⁶ cP, 10,000 cP and 30×10⁶ cP, 100,000 cP and 30×10⁶ cP or between 1×10⁶ cP and 30×10⁶ cP.

The biological tissue may comprise porcine tissue or human tissue.

The tissue scaffold may comprise a paste comprising the particles or granules of dermal tissue suspended in a liquid carrier, which may be as described above in relation to the first to fourth aspects of the invention, and in some embodiments the carrier liquid may be saline solution such as phosphate-buffered saline.

The use of particles or granules, such as a paste, comprising cross-linked tissue scaffold, in which the cross-linking agent is quercetin or genipin, confers particularly useful advantages. With genipin as the cross-linking agent the resultant tissue scaffold has a high mechanical (tensile) strength, stiffness and degree of cross-linking, which is particularly useful for applications in which such properties are paramount, such as large skin defects requiring structural reconstruction or where long term bulking is needed (sphincters e.g. to treat gastro—oesophageal gastric reflex disorder) is required. On the other hand, with quercetin as the cross-linking agent, the resultant tissue scaffold has a lower mechanical (tensile) strength, stiffness and degree of cross-linking, which is particularly useful for non-healing diabetic ulcers/chronic wounds or any type of fistula.

Utilising a paste or suspension, enables the tissue scaffold to be used in applications where it is crucial for the scaffold to either mould to an area to be repaired or to fill a wound, such as an area of skin injury.

The paste is both bioactive and biocompatible; it retains extra cellular proteins and surface proteoglycans which do not elicit an immune response in vivo but are crucial for optimum cellular interaction in vivo and tissue regeneration which is advantageous over prior art products which are not bioactive. Additionally the pastes of the invention are better able to integrate with the wound bed when compared to a known dermal sheet. By cross-linking the paste the beneficial molecular cues and signals are retained whilst also conferring biomechanical stability.

According to a sixth aspect of the invention there is provided a tissue implant comprising a tissue scaffold of the fifth aspect of the invention.

According to a seventh aspect of the invention there is provided a dermal replacement comprising a tissue scaffold of the fifth aspect of the invention.

According to an eighth aspect of the invention there is provided a method of manufacturing a tissue scaffold of the fifth aspect of the invention, the method comprising the steps of:
a) decellularising a biological tissue and cross-linking the biological tissue with quercetin and/or genipin;
b) preparing particles or granules of the biological tissue; and optionally
c) suspending the particles or granules in a carrier medium.

In some embodiments steps (a) and (b) are performed in order. In other embodiments steps (a) and (b) may be combined or step (b) may be performed before step (a). Decellularisation of the biological tissue in step (a) may be performed after cross-linking, or vice versa, but cross-linking is preferably performed after decellularisation.

In some embodiments steps (b) and (c) may be performed at the same time as step (a), or before step (a).

The tissue, quercetin and genipin may be as described hereinabove.

Step (a) may comprise subjecting the biological tissue to a decellularisation process comprising subjecting the tissue to osmotic shock. The decellularisation may comprise contacting the tissue sequentially with hypotonic and hypertonic solutions (in any order) to promote cell lysis.

The biological tissue may be immersed in the hypotonic and hypertonic solutions.

Contact, or immersion, of the biological tissue with the hypotonic and hypertonic solutions may be repeated at least once, and preferably at least twice, three times or four times.

The hypotonic solution may comprise sodium chloride. The hypertonic solution may further comprise ethylenediametetracetate (EDTA) and/or Tris-HCl.

The hypertonic solution may comprise between 0.5M and 2M NaCl, such as around 1M NaCl. The EDTA may be present at a concentration of between 10 mM and 100 mM, such as between 20 mM and 50 mM or around 25 mM. The Tris-HCl may be present at a concentration of between 20 mM and 100 mM, such as between 25 mM and 75 mM or around 50 mM.

The hypertonic solution may comprise 1M sodium chloride, as optimally 25 mM EDTA and 50 mM Tris-HCl.

The hypotonic solution may comprise EDTA and/or Tris-HCl which may be present at concentrations as described for the hypertonic solution described above.

The decellularisation process may also comprise contacting the tissue with one or more nuclease. The nuclease may be contacted with the tissue after the tissue is contacted with the hypotonic and hypertonic solutions. The nuclease may be a DNase or an RNase or a combination of a DNase and RNase. The decellularisation process may also comprise washing the tissue, preferably in a saline solution, such as phosphate buffered saline (PBS), after each step.

Step (a) may comprise contacting the decellularised tissue with quercetin and/or genipin at a temperature of at least 5° C., 10° C., 15° C. or 20° C., preferably ambient temperature, more preferably between 15° C. and 25° C.

Step (a) may comprise contacting the decellularised tissue with an aqueous solution of quercetin and/or genipin. The quercetin and genipin may be present in the aqueous solution at a concentration of at least 0.1 mg/ml, 0.2 mg/ml, 0.3 mg/ml or at least 0.5 mg/ml, such as at least 1 mg/ml or above.

Step (a) may comprise contacting the decellularised tissue with quercetin and/or genipin for a time period of at least 5 minutes, 10 minutes, 15 minutes, 30 minutes, 60 minutes, 120 minutes or 180 minutes.

The aqueous solution of quercetin and/or genipin may comprise further ingredients such as co-solvent, buffer components or the like, for example. In embodiments utilising quercetin, the quercetin may comprise quercetin in an ethanolic solution. The ethanolic solution may comprise at least 20% v/v ethanol, or at least 30% v/v ethanol or at least 40% v/v ethanol. In alternative embodiments the quercetin may be present in a saline solution, such as phosphate buffered saline (PBS) for example.

The decellularised biological tissue may be immersed in a solution of quercetin and/or genipin. The ratio of the volume of quercetin solution and decellularised tissue may be at least 0.1 ml/cm³, at least 0.2 ml/cm³ or at least 0.5 ml/cm³.

Step (b) may comprise grinding, milling, or cominuting a piece or sheet of biological tissue.

Step (b) may comprise forming particles or granules having a particle size of a diameter or length of at least 1 μm, 2 μm, 3 μm, 4 μm, 5 μm, 10 μm, 20 μm, 50 μm, 75 μm or 100 μm. In some embodiments the granules or particles may have an average diameter or length of 10 μm to 500 μm, 25 μm to 500 μm, 50 μm to 500 μm, 100 μm to 500 μm or 100 μm to 250 μm. The average particle size (diameter or length) may be measured using histological sections stained with Picro-sirius Red (PSR) and a graticule, or any other suitable method known to persons skilled in the art

There may be a step (d), after step (c) of washing the cross-linked tissue. Step (d) may comprise immersing the cross-linked tissue in a washing medium for at least 5 minutes, at least 10 minutes or at least 15 minutes. The cross-linked tissue may be subject to more than one washing step and step (d) may be repeated at least twice or three times. The washing medium may comprise a saline solution, such as PBS. The washing medium may comprise a preservative such as sodium azide, for example.

The biological tissue may initially be in the form of a sheet or piece of tissue. The sheet or piece of tissue may comprise dimensions of at least 1 cm×1 cm, 1 cm×2 cm, 2 cm×2 cm, 3 cm×3 cm or at least 4 cm×4 cm, for example.

There may be a further step of washing the particles or granules between step (a) and (b), between step (b) and (c) or between all steps of the process. The washing step may be repeated and may be as described for the fourth aspect of the invention.

The biological tissue may be as devised hereinabove for the fifth aspect of the invention, and may be dermal tissue, interstitial tissue, connective tissue or supporting tissue, but is preferably dermal tissue.

### DETAILED DESCRIPTION OF THE INVENTION

In order that the various aspects of the invention may be more clearly understood, embodiments thereof will now be described, by way of example, with reference to the accompanying drawings, of which:

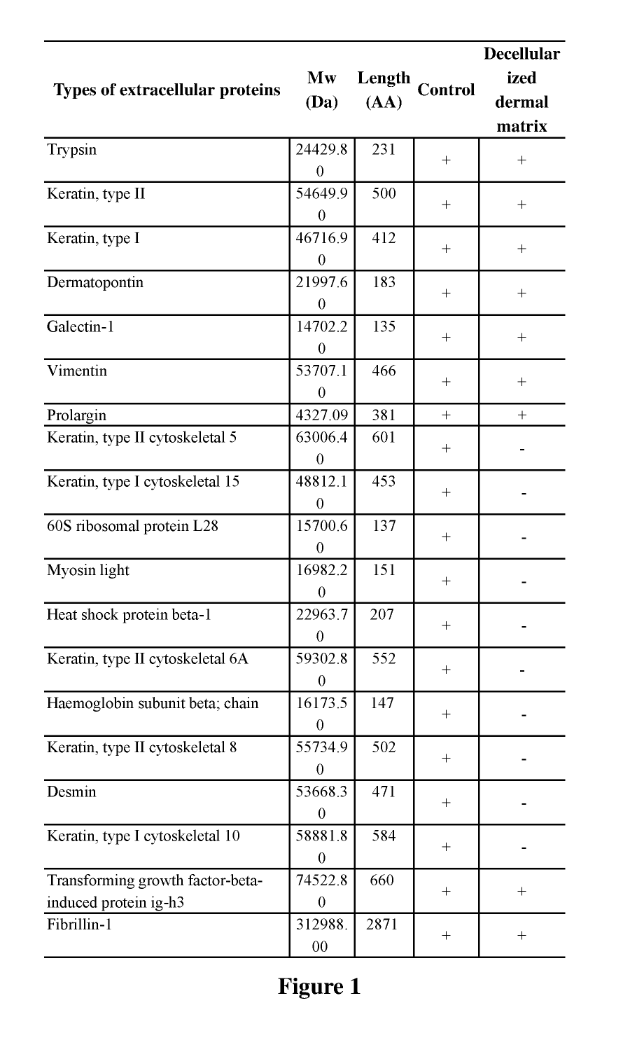

FIG. 1 shows mass spectrometry/proteomics analysis of extracellular matrix proteins in decellularised dermal tissue scaffolds of the invention after decellularization compared to a control (native dermis);

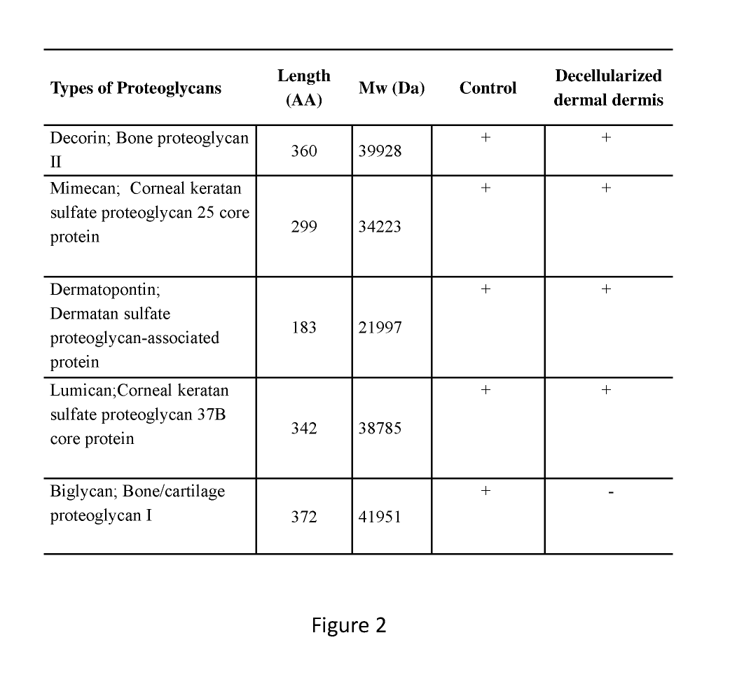

FIG. 2 shows mass spectrometry/proteomics analysis of proteoglycans present in the extracellular matrix after decellularisation compared to the control (native dermis);

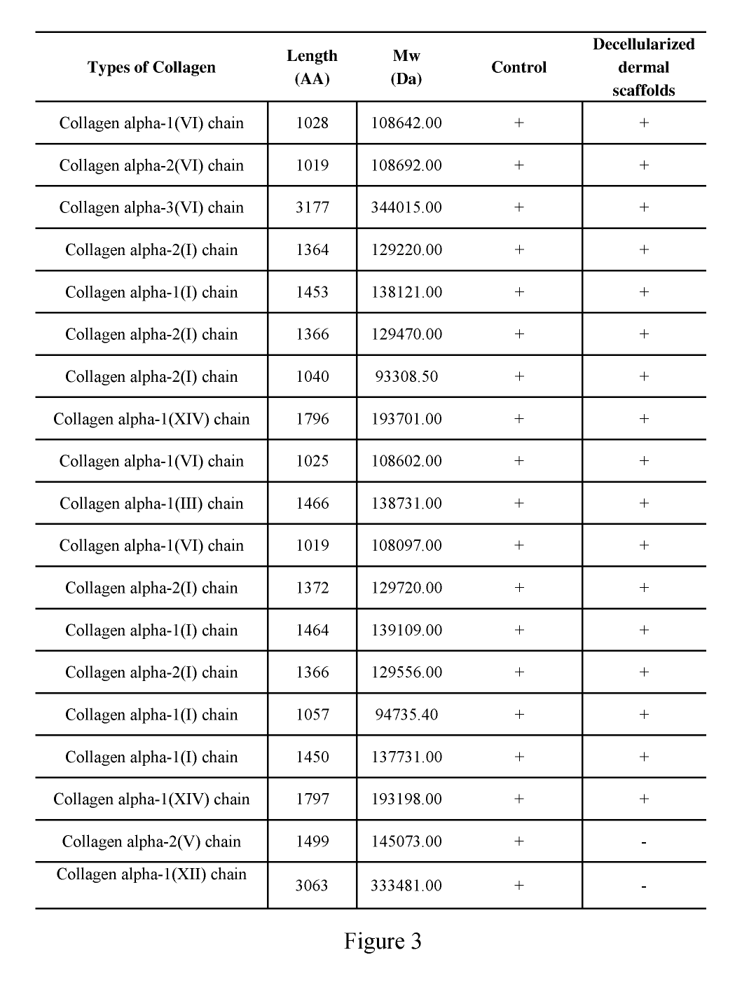

FIG. 3 shows mass spectrometry/proteomics analysis of the collagens present in the dermal scaffolds after decellularisation compared to control (native dermis);

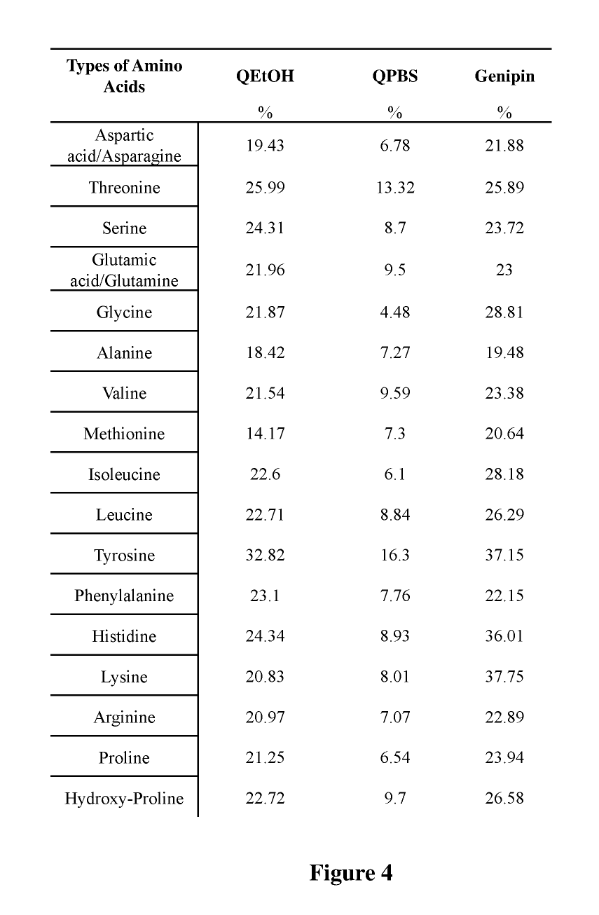

FIG. 4 shows the percentage of amino acid reduction in the decellularised dermal scaffolds after crosslinking with genipin and quercetin (dissolved in two solvents—PBS and EtOH);

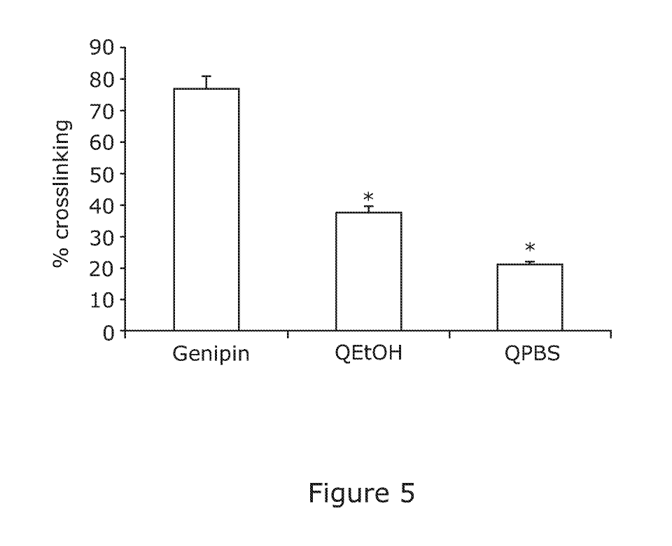

FIG. 5 shows the results of a ninhydrin assay used to determine the crosslinking degree of decellularised dermal scaffolds crosslinked with quercetin (EGEP dissolved in either PBS or EtOH) and genipin. *P<0.05 significantly different from genipin-crosslinked scaffolds;

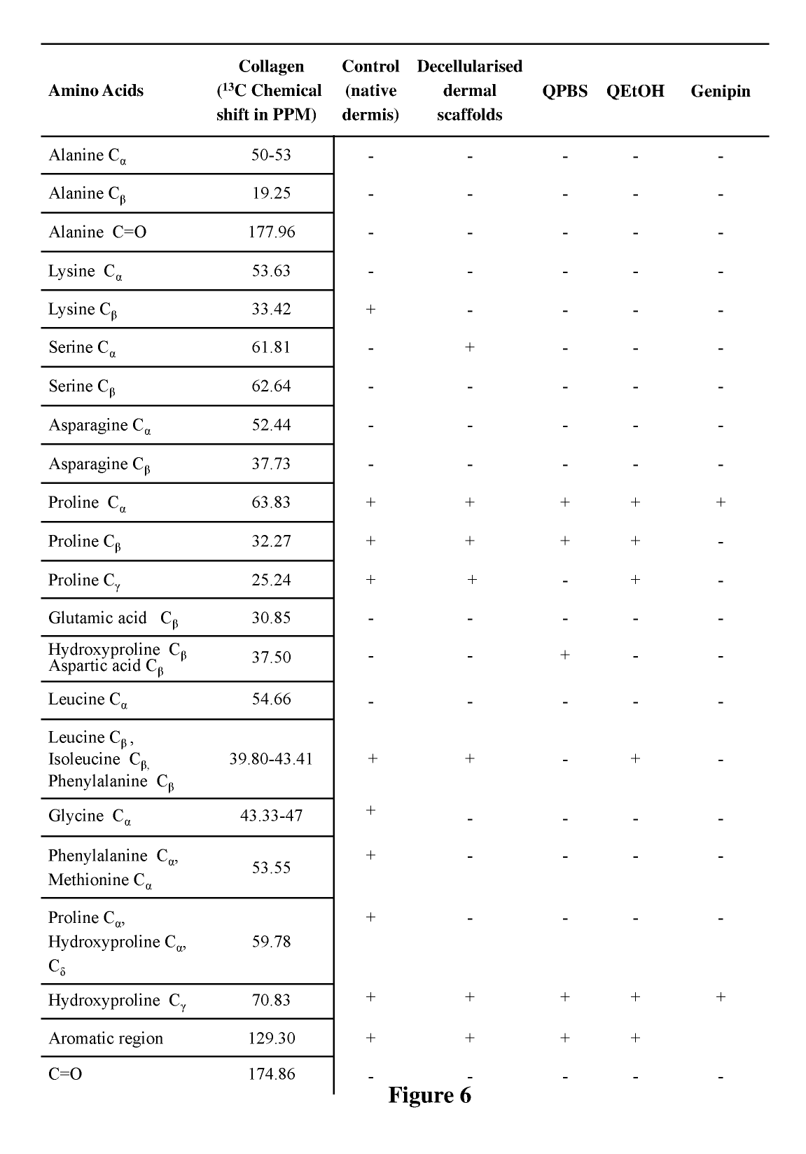

FIG. 6 shows the 13C chemical shift in PPM observed in the dermal scaffolds after crosslinking with genipin and quercetin (dissolved in two solvents—PBS and EtOH);

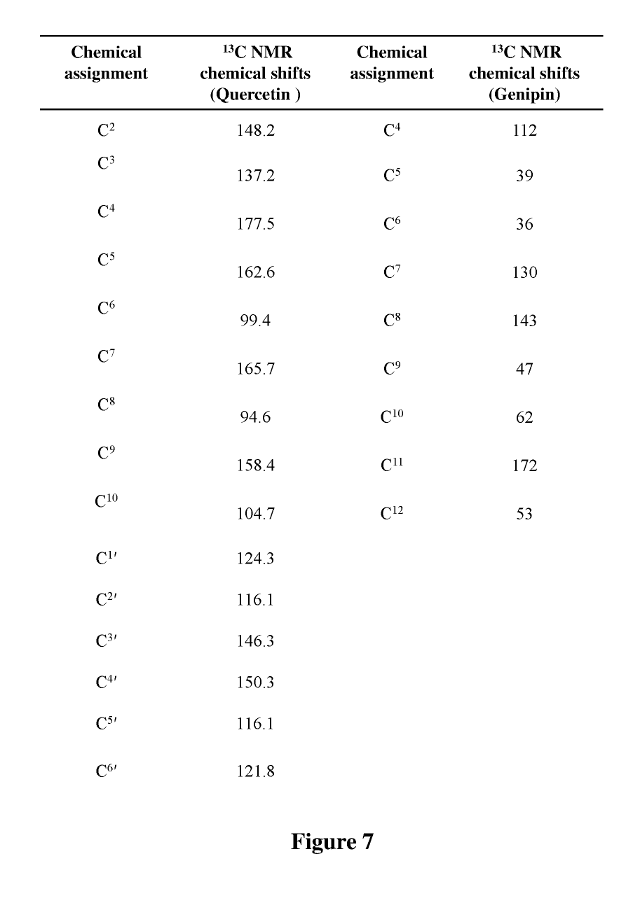

FIG. 7 shows the 13C chemical shift in PPM observed for the compounds quercetin and genipin used as crosslinking agents;

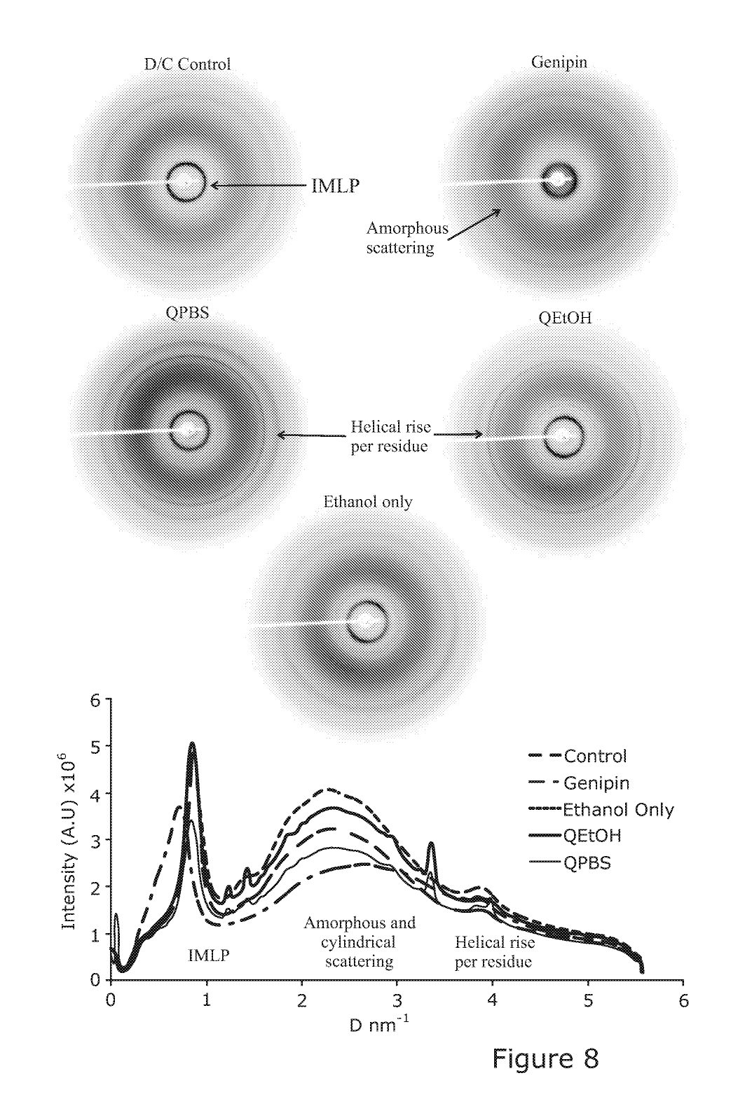

FIG. 8 shows the 2D-Wide Angle X-ray Diffraction (WAXD) patterns and corresponding 1D linear trace showing the intermolecular lateral packing (IMLP), amorphous and cylindrical scattering and helical rise per residue changes observed in the collagen fibrils diffraction treated under different conditions. Data are representative of three independent experiments;

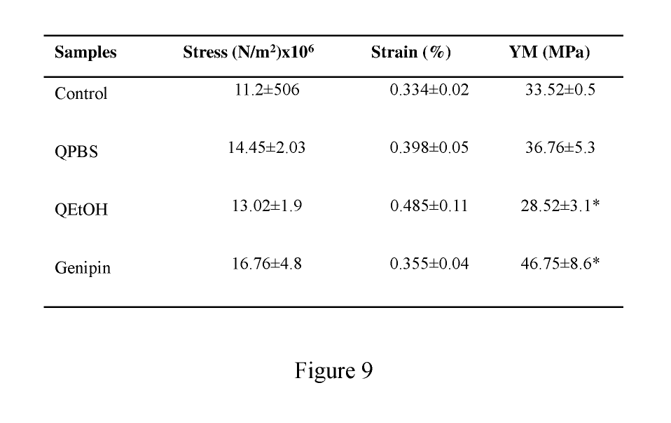

FIG. 9 shows a comparison of representative values of stress (N/cm2), strain (%) and the Young's modulus (YM; MPa). YM (ratio of stress over strain) was taken for comparison among samples: control (decellularised non-crosslinked dermal scaffolds), decellularised dermal scaffolds cross-linked with quercetin (dissolved in either PBS or EtOH) and genipin. *P<0.05 significantly different from control;

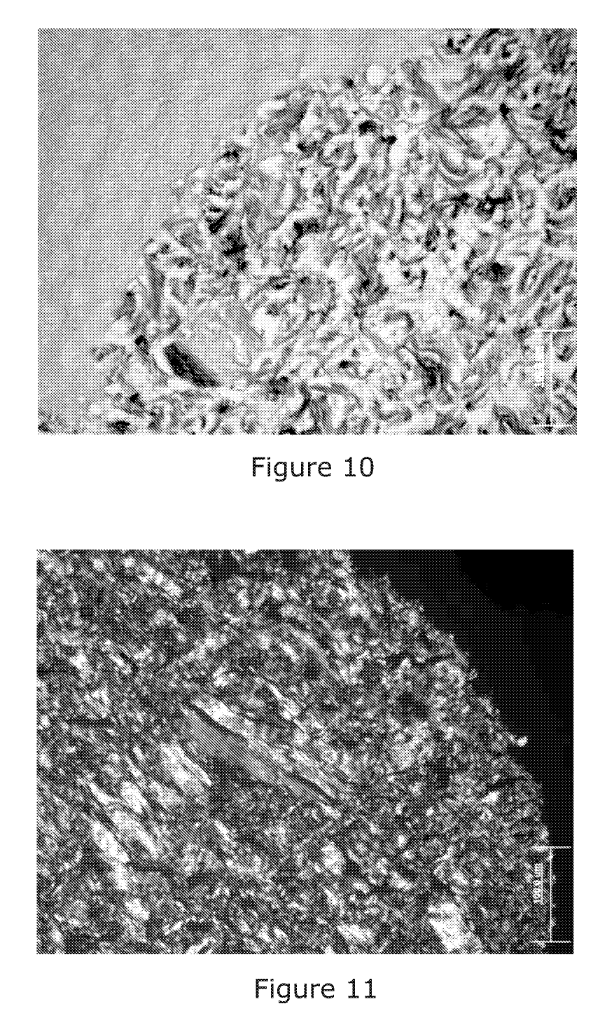

FIG. 10 is an image of a histological analysis, H&E section, of a decellularised paste prepared according to Example 2, using quercetin as the cross-linking agent; and

FIG. 11 is an image of a PSR section of the paste of FIG. 10 under polarised light showing collagen functional viability

### EXAMPLE 1—USE OF QUERCETIN AND GENIPIN TO CROSS-LINK DECELLULARISED DERMAL SCAFFOLDS

### 1. Materials and Methods

### 1.1 Dermal Scaffolds Crosslinking

Scaffolds were produced at Northwick Park Institute for Medical Research, London, UK. Fresh porcine skin was obtained from Large-White/Landrace crossbreed pigs. Skin was cleansed with soap, shaved and washed with water and iodine based solution (10% w/w Cutaneous Solution—Iodinated Povidone, Videne, Garforth, UK). The intact skin (epidermis and dermis) was dissected from the animal's flank, washed in sterile phosphate buffered saline (PBS, Sigma-Aldrich, Dorset, UK) with an antibiotic/antimycotic solution (AA; Sigma-Aldrich, Dorset, UK) five times and stored in sterile plastic bags at −20° C. for 24-48 h. The skin samples were defrosted, cut into pieces (2×2 cm), and allocated randomly into 2 groups for the production of decellularised scaffolds. Porcine acellular dermal scaffolds were produced using osmotic shock (by sequential application of hypertonic and hypotonic solutions) to provoke cell lysis, followed by multiple washing steps, in less than two days.

The decellularisation process is shown in the table below:

Protocol

Freezer (−20° C.) (24 h)
Distilled water (6 h)
Hypertonic solution (12 h) - 1M NaCl, 25 mM EDTA, 50 mM Tris-HCl
Wash buffer (8 h)
Hypotonic solution (12 h) - 25 mM EDTA, 50 mM Tris-HCl
Wash buffer (8 h)
Wash buffer (12 h)
Histological and molecular analysis

Samples were then allocated into 3 different groups (n=3 in each group) and crosslinked with either genipin (0.5% w/v in water; Wako Chemicals USA, Richmond, USA) or quercetin (1 mg/mL) for 3 h, under agitation, at room temperature. Preliminary tests were carried out to establish the best dose and time of crosslinking (data not shown). Quercetin was dissolved in two different solvents: ethanol 40% v/v (producing the QEtOH-crosslinked scaffolds) or PBS with 0.1% DMSO (QPBS-crosslinked scaffolds). The ratio of volume of the crosslinking agent and scaffold was 0.5 mL/cm². The crosslinked tissues were then thoroughly washed (4 washes of 10 min) with sterile PBS containing 1% sodium azide (Sigma-Aldrich, UK) and stored at 4° C. prior to all tests. Control samples were either non-decellularised native dermis or decellularised non-crosslinked dermal scaffolds, depending on the comparison for each test. All controls were stored under the same conditions for comparison.

1.2 Analysis of Extracellular Matrix Proteins after Decellularization

Protein extraction from tissues. Controls (non-decellularised dermis) and the decellularised dermal samples were homogenised on a beads beater at 6500 rpm for 45 seconds. Lysis buffer (8M urea, 50 mM Tris-HCL, pH 8.5, 5 mM DTT, 1% SDS, and protease inhibitor) was added to scaffolds and to get a final concentration of 20 mg/ml. The scaffolds were homogenized again with a beads beater at 6500 rpm for 45 seconds and repeated four times. The samples were centrifuged at 10,000 g for 5 min at 4° C. The supernatant was collected and protein concentration was determined by a BCA assay (Thermo, UK) and 100 ug of total proteins were added to a 30 kDa filter (Millipore, UK). Proteins were reduced by 20 mM DTT (Sigma, UK) at 37° C. for 1 h, and then alkylated with 100 mM iodoacetamide (IAA, Sigma, UK) for 45 min in the dark, at room temperature. Samples were centrifuged for 20 min at 14,000 g to remove DTT and IAA and followed by buffer exchange with 8 M urea once and 50 mM NH₄HCO₃ 3 times. One hundred microliter of trypsin was added at a trypsin/protein ratio of 1:50 for digestion at 37° C. overnight. Digested peptides were collected by upside down spin and washed twice with 0.5 M NaCl and water respectively. The peptides were purified by SepPak C18 cartridge (Waters, UK), dried in a SpeedVac and resuspended in buffer A (2% acetonitrile 0.1% formic acid) for LC-MS/MS.

Protein identification and quantitation by liquid chromatography-mass spectrometry (LC-MS). LC-MS/MS analysis was carried out by nano-ultra performance liquid chromatography tandem MS using a 75 μm-inner diameter×25 cm C18 nanoAcquity™ UPLC™ column (1.7-μm particle size, Waters). Peptides were separated with a 120 min gradient of 3-40% solvent B (solvent A: 99.9% H₂O, 0.1% formic acid; solvent B: 99.9% ACN, 0.1% Formic acid) at 250 nl/min and injected into a LTQ Orbitrap Velos (Thermo Scientific) acquiring data in positive ion mode. The MS survey was set with a resolution of 30,000 FWHM with a recording window between 300 and 2,000 m/z, a maximum acquisition time of 100 ms and the automatic gain control target set to 1,000,000 ions. Minimum MS signal for triggering MS/MS was set to 500, m/z values triggering MS/MS were put on a dynamic exclusion list (500 entries), and exclusion duration as 30 seconds. A maximum of 20 MS/MS scans were triggered per MS scan. The lock mass option was enabled and polysiloxane (m/z 371.10124) was used for internal recalibration of the mass spectra. All samples were measured in triplicate with the MS setting charge state rejection enabled and only more than 1 charge procures ions selected for fragmentation.

### 1.3 Amino Acid Analysis: Gas Phase Hydrolysis

To the sample (0.81 mg) in a pyrolysed tube was added norleucine (internal standard; 1004, of a 2.5 mM solution in 0.1M-HCl, 250 nmole), the tube was placed in a centrifugal evaporator and the mixture concentrated to dryness. Gas phase hydrochloric acid (constant boiling) hydrolysis was performed in an evacuated vial at 115° C. for 22 h. After removing traces of acid from the tube, sodium citrate loading buffer (pH 2.2) was then added to dissolve the residue. The resulting solution was filtered under centrifugation through a 0.2 micron filter. An aliquot of the filtrate (3%) was injected into an amino acid analyser (Biochrom 30 instrument) and chromatography was performed on an ion exchange resin (sodium system) eluting with a series of buffers over the pH range 3.2 to 6.45. Peak detection was achieved by mixing the eluate with ninhydrin at 135° C. and measuring the absorbance (at 570 and 440 nm). Quantitation was performed using Chromeleon® software and calibration curves for each amino acid of interest.

### 1.4 Determination of the Degree of Crosslinking: Ninhydrin Assay

The degree of crosslinking of the test samples (QPBS, QEtOH and genipin-crosslinked dermal scaffolds) was determined using the ninhydrin assay, and compared to control (decellularised, non-crosslinked dermal scaffolds). 50 mg of each sample was weighed, to which 1 mL of ninhydrin reagent (Sigma-Aldrich, UK), was added in clean test tubes. The contents of the tubes were vortexed and covered with aluminium foil and boiled for 2 min. The test tubes were then cooled and 1 mL of 50% ethanol was added to each test tube and the standards (glycine solution, Sigma-Aldrich, UK) and the absorbance was taken at 570 nm for the using a (Spectrostar Nano®, UK) spectrophotometer.

### 1.5 X-Ray Diffraction Analysis

Porcine dermal scaffolds control (non-crosslinked) and crosslinked samples (QPBS, QEtOH and genipin) were milled using a cryogenic mill (6770 Freezer/Mill®, SPEX SamplePrep, Stanmore, UK), yielding a fine dermal paste, which was allowed to dry (air-dry) for 24 hours prior to the tests. Collagen dried samples were packed into 25 μM transparent plastic polymer capilleries (MicroRT, Mitegen) and mounted on suitable cryo-bases. The samples were then exposed to X-rays using a microfocus rotating anode X-ray generator (Rigaku MicroMAX 007, Rigaku Europe, Kent, UK) source. The exposure times were 300 sec/image with a rotation of 10°. The resulting Wide Angle X-ray Diffraction (WAXD) images were recorded using a MAR345 image plate (Werkstr. 3, Norderstedt, Germany). Processing of the images to calculate 1 dimensional plot intensity profiles was carried out using the FibreFix software (CCP13; Diamond, Oxfordshire, UK).

### 1.6 Biomechanical Tests

Mechanical properties of control (decellularised non-crosslinked dermal scaffolds), genipin-, QPBS- and QEtOH-crosslinked scaffolds (n=3 for each group) were evaluated by tensile strength test measured as the maximum stress that each sample could withstand while being stretched before reaching breaking point. For each test, specimens were cut into dumbbell shape using a standard mould with a length of 2.5 cm and width of 0.4 mm. The tests were performed with the application of uniaxial tension using a tensiometer (Instron Inspec 220 Benchtop Portable tester, Instron, Buckinghamshire, UK). Samples were clamped into the holders and loaded at a constant tension rate. The tensile tester recorded the real-time load and elongation to which the tissue was subjected. Parameters such as maximum load (N), testing time (sec) and extension at maximum load (mm) were recorded. Strain was defined as rate of change of sample deformation and calculated as ratio of the length at the maximum load to the original length; and stress defined as how much load was applied at each meter squared sample. Young's modulus (YM; MPa) was calculated by the ratio of stress (N/m²) over strain (%).

### 1.7 Nuclear Magnetic Resonance (NMR) Spectroscopy

Structural characterization of control (native dermis), decellularised and crosslinked dermal scaffolds was performed using ¹³C NMR. Genipin and quercetin were dissolved in 0.5 mL of deuterated acetone for analysis of the compounds' ¹³C picks on their own. Prior to the NMR analysis the control (native dermis), decellularised non-crosslinked and crosslinked dermal scaffolds (genipin, QEtOH and QPBS) were digested in guanidine hydrochloride buffer (4M) for 3 h, transferred to a dialyzer with a NMWCO (12,000 Da) and dialyzed against distilled water overnight at 4° C. Twenty-five milligrams of these samples were then dissolved in deuterated chloroform (CDCl₃) and analysed on a Bruker AV400 (400 MHz) spectrometer. Spectra were analysed using the MestRec software package.

### 1.8 Statistics

Data were calculated as mean standard error, and significance was determined by performing two-tailed Student's t-tests or ANOVA (Prism 5: Graphpad Software, La Jolla, Calif.), followed by Bonferroni's post hoc test. A p value of less than 0.05 was considered to be significant.

### 2 Results

### 2.1 Mass Spectrometry (MS) and Protein Profiling

The protein profile of the porcine dermal scaffolds was assessed by MS and compared to control (native dermis) in order to assess the presence of the main ECM proteins known to take part in the crosslinking reactions and further tissue remodelling process. Results showed that after decellularisation, approximately 60% of the major extracellular proteins were retained in the matrix (as shown in FIG. 1). Proteoglycans such as decorin, mimecan, dermatopontin and lumican were not affected by the reagents used in the decellularisation process (as shown in FIG. 2). Similarly, approximately 90% of the different collagen types present in porcine dermis was retained after decellularisation (as shown in FIG. 3).

### 2.2 Amino Acid Analysis and Crosslinking Degree (Ninhydrin Assay)

The amino acid composition of genipin and quercetin-crosslinked dermal scaffolds was compared and shown in FIG. 4. The amount of amino acids was reduced in all crosslinked groups, and might be due to reaction with the crosslinking reagents. The highest percentage of amino acids reduction was observed with genipin. The amino acids that showed the highest reduction were lysine (37.75%), arginine (22.89%), asparagine (21.88%), glycine (28.81%) and alanine (19.48%). This reaction may potentially be occurring when quercetin dissolved in ethanol was used (QEtOH), which was shown to be more evident than in the QPBS-crosslinked scaffolds. The ninhydrin assay was used to determine the crosslinking degree of genipin and quercetin with the amino (aspartic acid/asparagine, threonine, serine, glutamic acid/glutamine, glycine, alanine, valine, methionine, isoleucine, leucine, tyrosine, phenylalanine, histidine, lysine and arginine) and imino (proline and hydroxyproline) groups within the porcine dermal scaffolds. Genipin-crosslinked scaffolds showed the highest degree of crosslinking (77.2±5%). Between the quercetin-crosslinked matrices, QEtOH showed the highest degree (37.6±1%) when compared to QPBS-crosslinked scaffolds (21.9±1%) as shown in FIG. 5.

### 2.3 X-Ray Diffraction (XRD) Analysis

Wide Angle X-Ray diffraction (WAXD) was used to capture the diffracting X-rays corresponding to changes in the packing features of collagen of the decellularized dermal scaffolds crosslinked under different conditions. FIG. 8 shows the 2D WAXD patterns and the corresponding linear intensity vs scattering vector trace generated from decellularised non-crosslinked (control), genipin-, QPBS- and QEtOH-crosslinked dermal scaffolds, as well as from scaffolds immersed in ethanol (EtOH; 40% v/v) only. In the WAXD patterns the equatorial reflection due to the intermolecular lateral packing (IMLP) arises from the interference function due to the lateral distance between nearest neighbor collagen molecules. The amorphous scattering region corresponds to the diffuse scattering of the non-crystalline regions of the collagen fibril and some scatter from the collagen helix; and the meridional reflection due to the helical rise per residue (seen in the edges) corresponds to the distance between polypeptide subunits within the polypeptide chain. The results showed that the packing features of collagen were altered only in the dermal scaffolds crosslinked with genipin, being evident as the peak that corresponds to IMLP in the scattering vector trace was shifted to the left (CT=0.855±0.006 Dnm⁻¹ vs G=0.73±0.003 Dnm⁻¹; p<0.00004), and the amorphous scattering curve was wider when compared to control samples. The packing features of the quercetin crosslinked scaffolds did not show any significant difference when compared to the control scaffolds.

### 2.4 ¹³C NMR Shifts

NMR study was carried out to investigate the molecular dynamics in the non-crosslinked dermal samples as well as the effect of crosslinking on the peptide backbone of the collagen fibril. The carbonyl signals (¹³C) of the non-decellularized porcine dermal samples (control) were clearly split into three peaks which are characteristic of the native structure in collagen fibrils. Most of the peaks arise from the amino acid residues such as proline, hydroxyproline and glycine (as shown in FIG. 6). Some peaks were readily assigned to amino-acid residues (phenylalanine and methionine), which were located in the partially denatured portion of the fibrils. However after decellularization, the ¹³C NMR signals broadened possibly due to hydration, while the amino acids such as proline, hydroxyproline and serine were still seen. The peaks of the non-collagenous amino acids such as glutamine, leucine, phenylalanine and methionine however were not readily seen in FIG. 6. In the genipin-crosslinked scaffolds, the amino acids peaks seen in the decellularised scaffolds were absent, as shown in FIG. 6, except for proline and hydroxyproline. The NMR peaks characteristic of genipin compound as listed in FIG. 7 were also absent in the genipin-crosslinked scaffolds. The NMR peak shifts of the quercetin-crosslinked scaffolds did not display any significant differences when compared to the non-crosslinked scaffolds as shown in FIG. 6. Most of the peaks arising from the amino acid residues such as proline and hydroxyproline were still seen in both quercetin-crosslinked matrices (QPBS and QEtOH). However, some other amino acids such as leucine, isoleucine and phenylalanine seen in the QEtOH were absent in the QPBS scaffolds. The NMR peaks characteristic of the quercetin compound as shown in FIG. 7, were not clearly visible in both quercetin-crosslinked scaffolds.

### 2.5 Biomechanical Tests

The stress-strain curves for the decellularised/non-crosslinked (control) and the crosslinked porcine dermal scaffolds are summarized in FIG. 9. As a result of crosslinking, the curves slope of collagen samples shifted towards the stress axis. The strain at the point of breakage was maximum for the QEtOH-crosslinked scaffolds and minimum for the genipin samples. The genipin-crosslinked dermal scaffolds, therefore, exhibited maximum tensile strength (46.75±8.6; p<0.01) while the QEtOH-crosslinked samples showed the lowest (28.52±9.1, p<0.05) when compared to controls.

### 3 Discussion

It is recognized that the majority of methods used for decellularisation can result in disruption of tissue architecture and potential loss of surface structure and composition compromising the ability of the scaffold to provide mechanical support during the remodelling process. The results described above for Example 1 show that a simplified decellularization method, designed to provoke an osmotic shock by using a combination of hypertonic and hypotonic solutions, without the use of enzymatic digestion is effective. After decellularization, the type of proteins, proteoglycans and collagens that were left behind in the ECM was assessed, as they are essential for successful crosslinking reactions and for cell-matrix interactions and tissue remodelling following implantation. The results show that decellularization had minimal effect on the extracellular proteins. The decellularised dermal matrices produced, showed absence of some of the cellular proteins (such as cytoplasmic cytoskeletal keratins, myosin and desmin) and also nuclear proteins (eg: ribosomal proteins) that were present in the native dermis (control). Glycoproteins and proteoglycans which facilitate the complex cell-cell and cell-matrix interactions were also retained following decellularization. Noteworthy is the preservation of small proteins that take part in the assembly of basement membrane such as prolargin and fibrillin. The latter not only helps the anchoring of basement membrane with surrounding tissue but also support elastic fibres deposition, which is essential for the viscoelasticity of the tissue. Together with fibrillin, dermatopontin may also be important after scaffold implantation as they can modulate the activity of the transforming growth factor beta (TGF-β), a critical growth factor important in the growth, proliferation and differentiation of cells. In addition, the results show that the main collagen types (I, II and III) were preserved after decellularization, and this is of major importance as the orientation of collagen fibres, can profoundly influence the directed migration of cells, possibly by potentiating growth factor receptor signalling or by mechanically reinforcing cell migration.

Although the effect of the decellularization reagents had minimal effect on the properties of the ECM scaffolds, it has been shown that the addition of exogenous crosslinking can markedly change the structure of the ECM. From the ninhydrin assay results, the highest degree of crosslinking was achieved when dermal scaffolds were treated with genipin and this was corroborated by the amino acid analysis. The amino acid analysis showed that a high number of primary amines such as lysine, hydroxyl-lysine, serine, glycine, alanine and arginine decreased, suggesting that they might be involved in the crosslinking reaction with genipin. However other amines such as glutamic acid, asparagine, threonine valine, methionine, isoleucine, leucine, tyrosine, phenylalanine, histidine, and proline/hydroxyproline were also reduced possibly indicating an involvement in the crosslinking reactions as well. The high number of amino groups shown to be involved in the crosslinking reaction corroborated the ninhydrin assay results, which showed that the highest degree of crosslinking was achieved when dermal scaffolds were treated with genipin. Quercetin compounds obtained from different sources may contain dihydrate, monohydrate or unhydrated forms, as well as a mixture of both hydrated and unhydrated forms. The crystal packing forces of quercetin seen in the dry state are absent when it is dissolved making it unstable in solution. Since a number of conformations may occur when quercetin is dissolved, two solvents were tested—an aqueous solution (PBS) and a polar solvent (ethanol 40% v/v—EtOH), and assessed also the different effects that these solvents may have on collagen crosslinking. The amino acid composition of the QEtOH and QPBS crosslinked scaffolds showed considerable differences. The percentage of reduced amino acids such as glycine, alanine, leucine, proline and hydroxyproline and other amino acids possibly involved in the crosslinking reaction were higher in the QEtOH-crosslinked scaffolds than in the QPBS scaffolds. This difference in the reduction of the amino acids between the QEtOH and QPBS scaffolds may be due to the nature of the solvent in which quercetin was dissolved. Quercetin is positively charged with abundant hydroxyl (OH) groups therefore number of interactions such as dispersion, dipole-dipole, hydrogen bonding and ionic interactions may occur between quercetin and the solvent. Similarly the collagen conformation and isoelectric points of the amino acids may change in the solvent thereby affecting their interactions with the crosslinking agent. Therefore when a polar solvent such as ethanol is used in the reaction, the effects on the conformation of the proteins are very complex. A possible reorientation of the amino groups of the collagen may take place and depending on their proximity to the structure of the crosslinking agent, the nature of their interaction is affected. For example the polar face of valine, alanine, leucine, methionine, tyrosine and phenylalanine interacts with the quercetin molecule comprised of two aromatic and a heterocycle ring, while lysine, arginine, histidine and asparagine are charged and involved in electrostatic interactions and serine, threonine and glutamic acid/glutamine involved in other non-covalent interactions. Therefore depending on the solvent used to dissolve quercetin, changes in the amino acids conformation could contribute to more than one interaction. These results were substantiated by the ninhydrin assay that showed that the degree of crosslinking in the QEtOH-crosslinked scaffolds was slightly higher than in the QPBS scaffolds. To further understand the alterations in the collagen structure, X-ray diffraction and NMR analysis were performed.

Wide Angle X-ray Diffraction (WAXD) was used to capture diffracting X-rays corresponding to the changes in the packing features of collagen such as intermolecular lateral packing (IMLP), amorphous and cylindrical scattering and helical rise per residue upon different conditions. Since the hierarchical arrangement of collagen is highly repetitive, any change in the distances between the collagen molecules is an effective way for measuring the alteration to the collagen molecular structure after crosslinking. IMLP, which is the distance between the collagen molecules in the lateral plane of the collagen fibril, showed to be altered only in the genipin-treated samples. Genipin molecules activate the functional groups of amines inducing an inter-crosslinking reaction, which may cause an increase in the distance between the collagen molecules in the lateral plane and therefore dislocating the peak that corresponds to IMLP in the scattering vector trace to the left. The peak corresponding to IMLP from the other samples remained unchanged when compared to the control samples. The distance between the amino acids along the polypeptide chains (helical rise per residue) for all other crosslinked samples remained unchanged when compared to the control. The non evident alteration in the collagen configuration might be due to a weak interaction between quercetin and collagen and also because the occurrence of the interactions between quercetin and collagen was probably low, showing that the binding of quercetin and collagen may not evidently alter the configuration of both quercetin and collagen.

On the other hand, the amorphous and cylindrical scattering of the genipin-crosslinked scaffolds were wider compared to control, which indicates a change in the crystallinity of the samples when genipin reacted with collagen molecule. No differences in the amorphous scattering of the quercetin crosslinked scaffolds were evident. Extra peaks for both QPBS and QEtOH scaffolds, which may be caused by diffraction of the highly crystalline structure of the unbound quercetin compound were also observed.

¹³C NMR is another analytical tool known for its sensitivity to the isotropic chemical shifts of carbon atomic resolution in proteins both before and after crosslinking. The peak positions of several major and minor amino acids obtained from the ¹³C NMR studies were compared in the non-decellularised samples (control), decellularised non-crosslinked, and in the three crosslinked dermal scaffolds (genipin, QPBS and QEtOH). Control samples exhibited prominent peaks for both major and minor amino acids similar to that of native type I collagen as well as peptides and proteins. However, after decellularization, the intensity of the peaks for some amino acids such as alanine, glycine, leucine, phenylalanine, methionine, serine, lysine and arginine were either too low for detection or obscured by the intense peaks of the major amino acids. It is also possible that the conformation of these amino acids may have become distorted during the decellularization process. Type I collagen is a triple helix chain and often steric hindrances in the helix can cause a reorientation of the peptide backbone, amino acid sequence, differences in the crystal chain packing and molecular conformation. Therefore possible steric hindrances and partial conformational denaturation during decellularization may have caused variations in the NMR shifts between the control and decellularised scaffolds. The peaks of the quercetin-crosslinked scaffolds showed some minor changes. This difference is likely to be due to the effect of hydration and dehydration caused by the solvents. The QEtOH-crosslinked scaffolds underwent dehydration due to which there were changes in the molecular packing within or amongst the triple helical chains of the collagen compared to the QPBS scaffolds. As a result shifts in the ¹³C peaks that are generally sensitive to the conformational changes were observed. For instance, proline C_{γ}, hydroxyproline C_{γ}, leucine, isoleucine and phenyalanine are present in the QEtOH-scaffolds but not in the QPBS scaffolds. The genipin-crosslinked scaffolds, on the other hand, showed no distinct peaks for the amino acids that were previously observed in the non-crosslinked scaffolds. Some studies have suggested that conformational changes from αhelix to a β sheet in silk fibroin sheets is known to occur when crosslinked with genipin. Amino acids in the collagen fibrils such as the polyalanine sequences are interspersed and ambivalent in nature switching from random coil, α-helix or a β-strand depending upon several factors one of which is the crosslinking solutions used. The absence of the major peaks in the genipin-crosslinked samples may be due to the reorientation of the amino acids switching from random coil, α-helix or a β-strand but due to their low intensity, the peaks were not visible or it could also be possible that these amino acids were taken up in the crosslinking reaction. Finally since the changes in the chemical structure of the dermal scaffolds after crosslinking with either genipin or quercetin may interfere with the mechanical properties of the scaffolds, the physical properties of the dermal scaffolds using Young's modulus test (YM; ratio of stress over strain) test were assessed. The highest YM value was observed for the genipin-treated scaffolds. The major factors impacting the mechanical properties of the dermal scaffolds are the ratio and the spatial orientation of the amino groups in the molecular chains of the collagen. With the increase in the ratio of the amino groups in the molecular chain, the number of crosslinking points is increased which in turn affects the crosslinking density. The chemical nature of the crosslinking agent used may also have an effect on the crosslinking density, for example some amino acid residues on the collagen that is normally far apart for crosslinking agents that are shorter in length become available to those crosslinking agents that can form crosslinks of various lengths thereby increasing the crosslinking density. In addition, the tensile strength of the collagen crosslinked scaffolds may vary depending upon the energy and strength required to break the type of bonds formed after crosslinking. It is known that the carbon bonds (—C—C—) are weaker than the disulphide bonds (—S—S—) introduced by some crosslinking agents such as dimethyl suberimidate, as results the tensile strength of the crosslinked scaffolds reduced from 9.50 MPa to 5.40 MPa. Therefore the highest tensile strength of the genipin crosslinked scaffolds may be correlated to an increased crosslinking density as well as the bond strength which resisted deformation when compared to the quercetin-treated scaffolds.

Ideally biological scaffolds should have a preserved ECM accompanied by a degradation rate that can keep pace with the speed of tissue regeneration and this may be possible by tailoring the exogenous crosslinking reactions. These results show that the flavonoid quercetin is a useful crosslinking agent for dermal scaffolds and dermal replacements.

### EXAMPLE 2—MANUFACTURE OF A PASTE COMPRISING QUERCETIN AND GENIPIN CROSS-LINKING

### 4.1 Dermis Harvesting

Fresh porcine skin was obtained from Large-White/Landrace crossbreed pigs in a clean environment. The skin was cleaned with soap, shaved and washed with warm water. An Iodine based solution (10% w/w Cutaneous Solution—Iodinated Povidone, Videne, Garforth, UK) was applied followed by a rinse with sterile PBS. A layer of the dermis (approximately 1 mm thick) was removed using a dermatome (Air dermatome, Zimmer, Ind., USA). Samples were cut (3×3 cm) using a mould cutter and then washed in sterile PBS (PBS, Sigma-Aldrich) with 2% antibiotic/anti-mycotic solution (AA; Sigma-Aldrich, Dorset, UK) five times. Samples were stored in sterile plastic bags at −20° C. for 24 h as part of a decellularisation process.

### 4.2 Decellularisation Process

Porcine dermis samples were defrosted and decellularised using hypertonic and hypotonic solutions followed by multiples washing steps as described above in section 1.1 of Example 1.

### 4.3 Dermal Paste Production

Following decellularisation, dermal sheets were used to create a collagen paste. Briefly the dermal scaffold sheets were finely cut into small pieces (˜2 mm) and placed into vials prior to cryo-milling. Two grams of dermal scaffolds were cut and cryo-milled in order to obtain homogeneous particles size between 100-250 μm. A consistent paste was achieved when the following protocol was used: pre-cooling 5 min, 1 cryo-milling cycle (rate:10 cps) for 2 min.

The collagen fragments (paste pre-cursor) were removed from the cryo-milling vial, placed in a 50 mL falcon tube and washed with sterile PBS, followed by centrifugation (2000 g, 5 min); this process was repeated 3 times. To crosslink the paste, a crosslinking solution (either 0.5% genipin in water; quercetin 1 m/mL in PBS or EtOH 40% v/v) was added to the paste (0.5 mL/gram of paste) and left for 3 h under agitation. The crosslinking solutions were removed by centrifugation and paste was washed with sterile PBS 3 times, followed by centrifugation (3500 g for 20 min). Resulting paste pellets were diluted with PBS 4:1 (v:v) to form a 80% paste.

Physical parameters of the pastes were tested in accordance with the protocols of sections 1.2-1.7 of Example 1 above and similar results were achieved. In particular the pastes produced according to the method of the invention of Example 2 were found to be excellent dermal implants and dermal replacement tissue, with the quercetin cross-linked pastes forming a more flexible scaffold and the genipin cross-linked pastes forming a less inflexible scaffold. FIGS. 10 and 11 show histological analysis (hematoxylin and eosin staining (H&E) and Pico-Sirius Red (PSR) staining respectively) of the quercetin cross-linked paste, showing that it is decellularised (FIG. 10) and has viable collagen functionality (FIG. 11).

The above embodiments are described by way of example only. Many variations are possible without departing from the scope of the invention as defined in the appended claims.

1. A tissue scaffold comprising particles or granules of biological tissue, wherein the biological tissue is cross-linked with quercetin and/or genipin.

2. A tissue scaffold as claimed in claim 1, wherein the biological tissue is selected from dermal tissue, interstitial tissue, connective tissue or supporting tissue.

3. A tissue scaffold as claimed in claim 2, wherein the biological tissue comprises dermal tissue.

4. A tissue scaffold as claimed in claim 1 wherein the granules or particles comprise a paste.

5. A tissue scaffold as claimed in claim 1 wherein the granules or particles are suspended in a carrier medium.

6. A tissue scaffold as claimed in claim 5 wherein the granules or particles are suspended in a carrier medium at a concentration of at least 50% w/v.

7. A tissue implant or dermal replacement comprising a tissue scaffold of claim 1.

8. A method of manufacturing a tissue scaffold comprising the steps of:
(a) decellularising a biological tissue and cross-linking the biological tissue with quercetin and/or genipin.
(b) preparing particles or granules of the biological tissue; and optionally
(c) suspending the particles of granules in a carrier medium.

9. A method as claimed in claim 8, wherein steps (a) and (b) are performed in order.

10. A method as claimed in claim 8 wherein step (b) is performed before step (a).

11. A method as claimed in claim 8 wherein decellularisation of the biological tissue in step (a) is performed before cross-linking.

12. A method as claimed in claim 8 wherein decellularisation of the biological tissue in step (a) is performed after cross-linking.

13. A method as claimed in claim 8 wherein step (c) is performed before step (a), or before step (b).

14. A method as claimed in claim 8 wherein step (a) comprises subjecting the biological tissue to osmotic shock.

15. A method as claimed in claim 8 wherein the decellularisation comprises contacting the biological tissue sequentially with hypotonic and hypertonic solution, in any order.

16. A method as claimed in claim 15, wherein contact with the hypotonic and hypertonic solutions is repeated at least once.

17. A method as claimed in claim 8 wherein cross-linking of the biological tissue with quercetin and/or genipin is performed at a temperature of between 15° C. and 25° C.

18. A method as claimed in claim 8 wherein the quercetin and/or genipin comprises an aqueous solution comprising a concentration of quercetin or genipin of at least 0.1 mg/ml.

19. A method as claimed in claim 8 wherein cross-linking with quercetin and/or genipin is performed for at least 5 minutes.

20. A method as claimed in claim 8 wherein step (b) comprises forming particles or granules having an average particle size of 10 μm to 500 μm.

21. A method as claimed in claim 8 further comprising washing the biological tissue after step (a), after step (b) or after step (c), or after any two or more of steps (a), (b) and (c).

22. A method as claimed in claim 8 wherein the biological tissue comprises dermal tissue, interstitial tissue, connective tissue, or supporting tissue.

23. A method as claimed in claim 8 wherein the quercetin comprises an ethanolic solution of quercetin or an aqueous solution of quercetin.

24. A method as claimed in claim 23 wherein the ethanolic solution comprises at least 20% v/v ethanol.

25. A method as claimed in claim 23 wherein the aqueous solution comprises phosphate buffered saline solution.

26. A tissue scaffold comprising decellularised dermal tissue cross-linked with quercetin.

27. A tissue scaffold as claimed in claim 26 wherein the dermal tissue comprises both epidermis and dermis.

28. (canceled)
